(19) United States
(12) Patent Application Publication
(10) Pub. No.: US 2008/0125787 A1
(43) Pub. Date: May 29, 2008

(54) Dynamic rod

(76) Inventors: Robert L. Doubler, Monroe, MI (US); John E. Hammill, Maumee, OH (US)

Correspondence Address: MCHALE & SLAVIN, P.A., 2855 PGA BLVD, PALM BEACH GARDENS, FL, 33410, US

(21) Appl. No.: 11/938,875

(22) Filed: Nov. 13, 2007

Related U.S. Application Data
(60) Provisional application No. 60/867,333, filed on Nov. 27, 2006.

(51) Int. Cl. A61B 17/58; A61B 17/08

(52) U.S. Cl. 606/103; 606/151

(57) ABSTRACT

Disclosed is a dynamic rod assembly for intervertebral stabilization that allows for load sharing across vertebrae by providing predetermined compression, distraction and bending. The dynamic rod is based on a first and second rod having a flexible union. The flexible union includes a retaining element for securing the rods in position and a bearing element interposed between a first and second bearing seat permitting the compression, distraction and bending.

### RELATED PATENTS

This invention claims the priority date of provisional patent application No. 60/867,333 filed Nov. 27, 2006 the contents of which is incorporated herein by reference.

### FIELD OF THE INVENTION

This invention is related to cervical spine surgery and in particular to a dynamic rod used for posterior pedicle fixation.

### BACKGROUND OF THE INVENTION

The most common type of posterior pedicle fixation for vertebral bodies within a human spine is performed by use of pedicle screws, hooks and the like which are attached to vertebrae and then secured together with rods or plates for immobilization and stabilization of the vertebral bodies. Such systems are generally rigid and do not accommodate load sharing across multiple spinal vertebrae occurring through either physical activity or attribute of the individual or characteristics due to aging.

Spinal fixation, such as lumbar sacral fusion and the correction of spinal deformities such as scoliotic curves, is a well known medical procedure. Pedicle, lateral, and oblique mounting devices may be used to secure corrective spinal instrumentation to a portion of the spine that has been selected to be fused by arthrodesis. Spinal fixation systems typically include corrective rods attached to selected vertebrae of the spine by screws, hooks, and clamps. The corrective rods are generally parallel to the patient's back and may include transverse connecting rods that extend between neighboring spinal rods. Spinal fixation systems are used to correct problems in the lumbar and thoracic portions of the spine, and are often installed posterior to the spine on opposite sides of the spinous process and adjacent to the transverse process.

U.S. Pat. No. 5,222,954 discloses a spinal implant that has an adjustable rod to connect and stabilize the vertebrae of a vertebral column. The device employs a clamp that is placed over the screw having a bore that is larger than the major diameter of the screw, so that the clamp can float about the screw. The clamp has a second bore perpendicular to the first bore, adapted to receive and hold a spinal rod.

U.S. Pat. No. 5,645,599 discloses an interspinous stabilizer having members which are to be anchored to the spinous processes and which are joined together by a U-shaped leaf spring in a plane perpendicular to the geometrical generatrix of the spring.

U.S. Pat. No. 5,415,661 discloses a device that includes a curvilinear rod designed to provide a specified amount of flexibility, such that the implant supposedly restores normal biomechanical function to the vertebrae of the spine receiving the implant. The rod is limited to a curvilinear shape that has a radius of curvature of between 0 to 180 degrees.

U.S. Pat. No. 6,293,949 discloses a flexible spinal stabilization device that includes a longitudinal portion that includes a series of shapes that have an accordion appearance. The device is intended for use along the cervical vertebrae, and it is intended to be installed along the anterior side of the vertebrae.

U.S. Pat. No. 6,440,169 discloses a device that attaches to the spinous processes of two vertebrae and has a leaf spring that allows the device to compress and then recover spontaneously after the stress has ceased.

U.S. Pat. No. 7,238,204 discloses an intervertebral implant having two opposite notches against which the spinous processes come into abutment. The notches present relative mobility and relative movements between them are damped. The intervertebral implant, a spacer, is designed to be applied between two spinous processes of two vertebrae, the implant having a connection piece made of a material having elastic deformability so that the stresses that are exerted on the elements are damped. The implant also limits the relative movements of the vertebrae.

What is needed is a dynamic rod for use in posterior pedicle fixation that provides a load sharing across more than one vertebra by facilitating limited compression, rotation, distraction and bending.

### SUMMARY OF THE INVENTION

Disclosed is a dynamic rod for use in posterior pedicle fixation that allows for load sharing across vertebrae by providing predetermined compression, distraction and bending. The dynamic rod enables cyclical loading of the motion segment unit through dynamic fixation and enhances load sharing between the inter body device and the vertebral body implants. The result is a dynamic rod capable of reducing the stress on a pedicle screw to bone interface.

The dynamic rod is based on a first rod, a second rod and a flexible union for connecting the first and second rod. Each rod has an enlarged end portion operatively associated with the flexible union including a cup member being generally cylindrical in shape and having a first open end and a second open end. The enlarged portion of the first rod is larger than the first open end of the cup member and the enlarged portion of the first rod bears against an inner surface of the cup member. The enlarged portion of the second rod is smaller than the second open end of the cup member and the enlarged portion of the second rod bears against an inner surface of the cup member.

The union includes a retaining element secured to the second opening of the cup member to prevent disengagement of the enlarged portion of the second rod from the cup member. The flexible union includes a bearing element interposed between a first bearing seat for the bearing element and a second bearing seat for the bearing element and a first resilient member interposed between the first bearing seat and an end face surface of the enlarged portion of the first rod and a second resilient member interposed between the second bearing seat and an end face surface of the enlarged portion of the second rod.

Thus, an objective of the invention is to provide a dynamic rod assembly for intervertebral stabilization.

Still another objective of the invention is to provide a dynamic rod for use in posterior pedicle fixation.

Another objective of the invention is to provide a load sharing across more than one vertebra by facilitating limited compression, rotation, distraction and bending.

Still another objective of the invention is to disclose a dynamic rod capable of providing predetermined compression, distraction and bending.

Yet still another objective of the invention is to disclose a dynamic rod that enables cyclical loading of the motion segment unit through dynamic fixation and enhances load sharing between the inter body device and the vertebral body implants.

Still another objective of the invention is to provide a dynamic rod capable of reducing the stress on a pedicle screw to bone interface.

Yet still another objective of the invention is to provide a dynamic rod assembly for intervertebral stabilization based upon various embodiments of a ball bearing riding on bearing seats definitively retained by O-rings, flexible washers, and springs thereby enabling intervertebral compression, distraction, and flexation.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

FIG. 1 is a pictorial view of the dynamic rod indicating a range of motion;

FIG. 2 is a pictorial illustration of a dynamic rod placed between two pedicle screws providing a union for dynamic motion and a third pedicle screw set in a rod for a fixed position;

FIG. 3 is a side view of the dynamic rod of the instant invention;

FIG. 4 is a cross sectional portion of the union depicted in FIG. 3;

FIG. 5 is a cross section of the union shown in FIGS. 3 and 4;

FIG. 6 is an exploded view of a first portion of the dynamic rod;

FIG. 7 is a view of the second portion of the dynamic rod securable to the first portion shown in FIG. 6;

FIG. 8 is a view of the spherical bearing placed between the dynamic rod sections;

FIG. 9 is an exploded view of one embodiment of the instant invention;

FIG. 10 is a perspective view of the cap nut;

FIG. 11 is a perspective view of the cap o-ring;

FIG. 12 is a perspective view of the cap;

FIG. 13 is a perspective view illustrating laser welding of the cap;

FIG. 14 is a second embodiment of the dynamic rod employing springs;

FIG. 15 is a perspective view of a Belleville spring washer;

FIG. 16 is a cross sectional view of the Belleville spring washer shown in FIG. 15; and

FIG. 17 is a perspective view of a spring used in the second embodiment.

### DETAILED DESCRIPTION OF THE PREFERRED EMBODIMENT

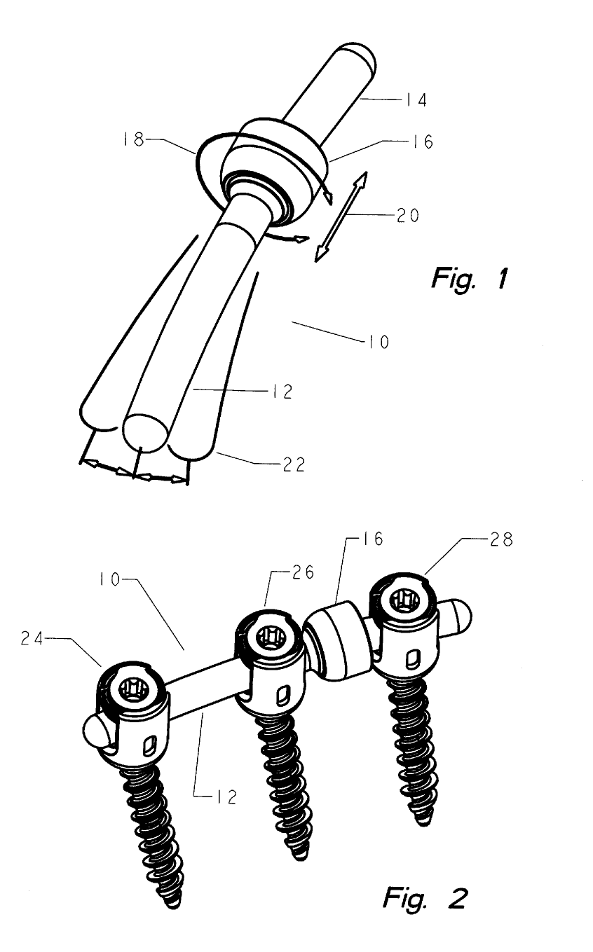

Now referring to the Figures in general and FIG. 1 in particular, depicted is the dynamic rod (10) illustrating a range of motion wherein a first rod (12) is coupled to a second rod (14) by use of the union (16). The union preferably allows a 360° range of motion (18) as well as longitudinal compression, and distraction (20). Angular deflection is infinite within a predetermined conical range depicted by (22). FIG. 2 depicts a dynamic rod (10) in a two level construct wherein a solid rod is shown between pedicle screws (24, 26) being the lower portion of the first end (12) with the union (16) depicted between the second pedicle screw (26) and a third pedicle screw (28).

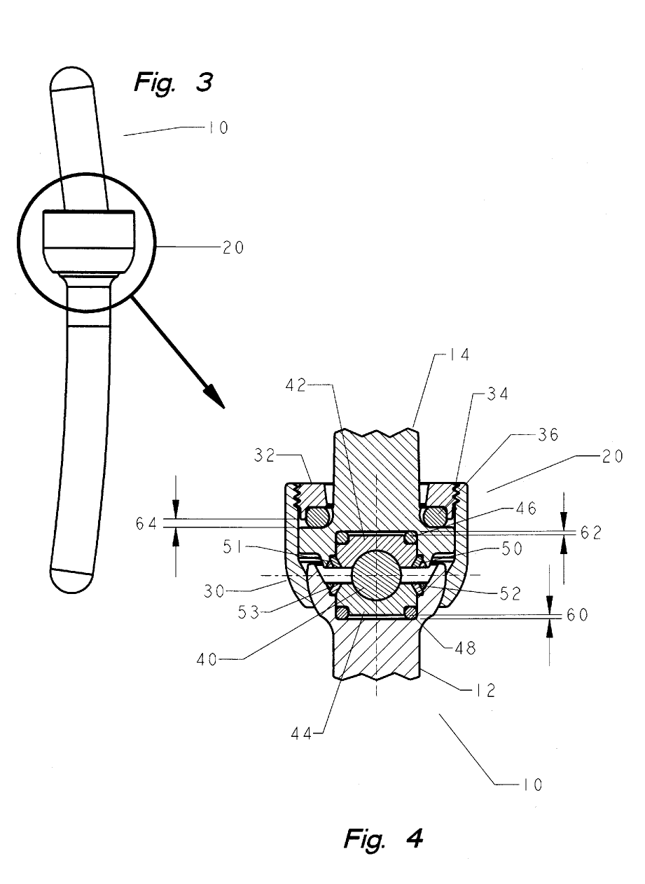

FIG. 3 depicts the dynamic rod (10) with a cross sectional portion of the union (20) as set forth in FIG. 4. FIG. 4 is a cross sectional view of the dynamic rod (10) and in particular the union (20). The first rod (12) is connected to the second rod (14) wherein a cap nut (30) is shown threadably engaged with threaded ring (32). Threaded ring (32) may be welded to the cap nut illustrated as weldment (34). The result is a dual-ball-in-socket device having an o-ring (36) constructed of a suitable polymer or the like to provide a spring/sponge type motion for purposes of distraction and rotation. Between the first and second rod is a ball bearing (40) placed in ball seats (42, 44) isolated from the rods 12 and 14 by O-rings (46, 48). O-rings (46, 48) are made of polymeric materials and provide the angular compressive movement. Preferably all components (other than the O-rings) are made of titanium or stainless steel materials and may include various coatings well known in the art. The spherical ball and cooperating seats could also be made from a ceramic material without departing from the scope of the invention. The seats (42, 44) are preferably held in place by retaining rings (51, 53) which may be secured by weldments (50, 52) or other suitable means, well known in the art, of securing the retaining rings to the rods. Compression motion is completed by compressing o-ring (48) having a space component depicted by (60) and o-ring (46) having a space component depicted by (62) upon reaching the predetermined limit the space components will be closed. Similarly distraction motion is completed by compressing O-ring (36) having a predetermined space component depicted by (64) upon reaching the predetermined limit the space components will be closed.

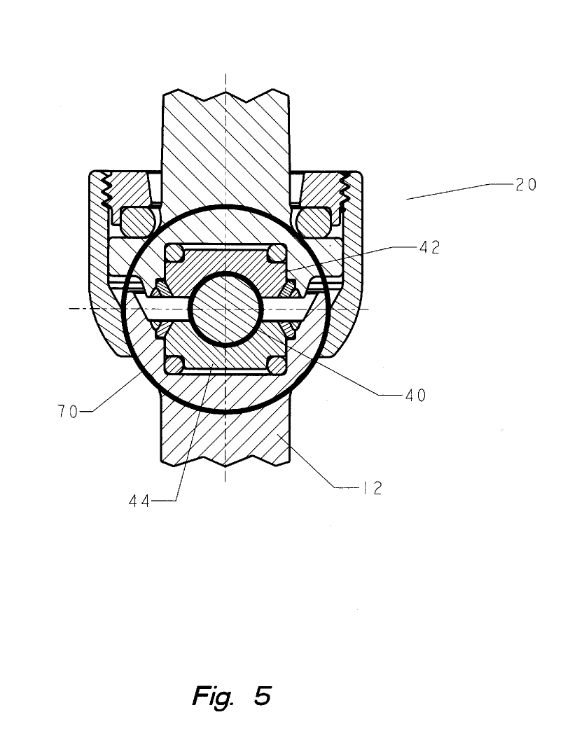

FIG. 5 refers to union (20) with an emphasis on the ball-in-socket concept having ball bearing (40) within seats (42, 44). The bold ring depicted by (70) illustrates the spherical segment integrally formed on the end of the first rod (12) used for providing pivotal flexation within a predetermined range.

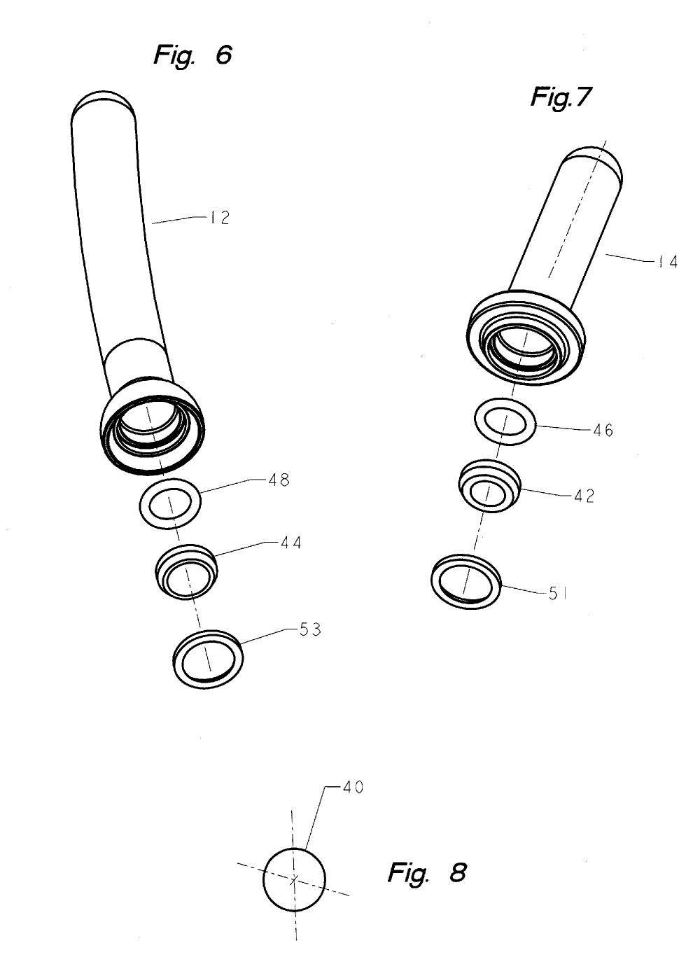

FIGS. 6, 7 and 8, show exploded views of the components of both the first rod (12) and the second rod (14) with the O-ring (48) held in position by seat member (44) and retaining ring (53). The ball bearing (40) is constructed and arranged for a substantially conjugate fit into seat (44). Similarly the illustrated second rod (14) components include O-ring (46) which is held in place by seat (42) both of which are contained by retaining ring (51).

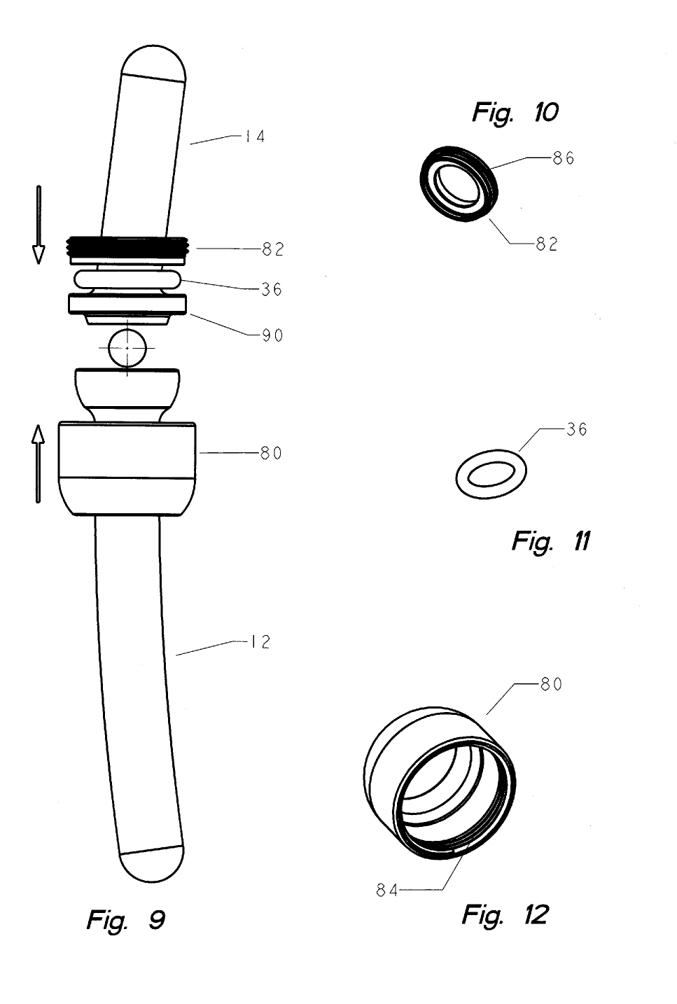

FIG. 9 illustrates assembly of the components depicted in FIGS. 6, 7 and 8 with first rod (12) being adjoined to second rod (14) by use of a cap (80) operatively associated with and securable to a cap nut (82). The O-ring (36) is positioned between the cap nut (82) and housing component (90) of the second rod (14) for longitudinal distraction and pivotal flexation.

FIG. 10, shows the cap nut (82) having external threads (86).

FIG. 11 illustrates the O-ring (36)

FIG. 12 depicts the cap (80) which includes internal threads (84)

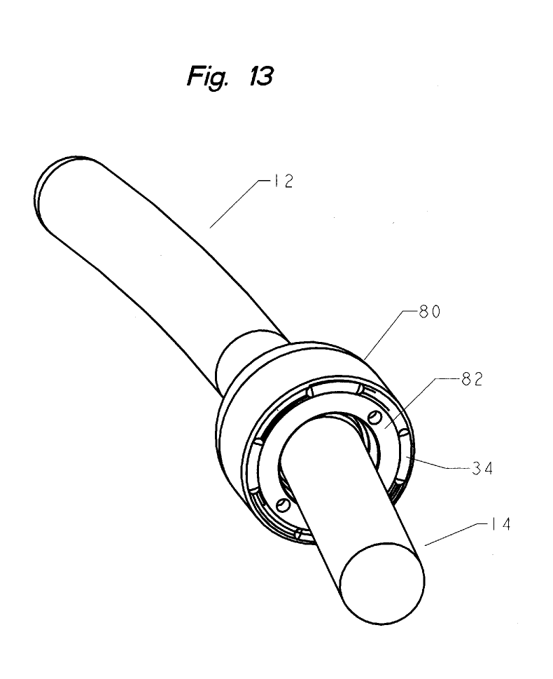

FIG. 13 is a view of the cap (80) secured to the cap nut (82) which is then laser welded, producing a weldment (34) to prevent separation.

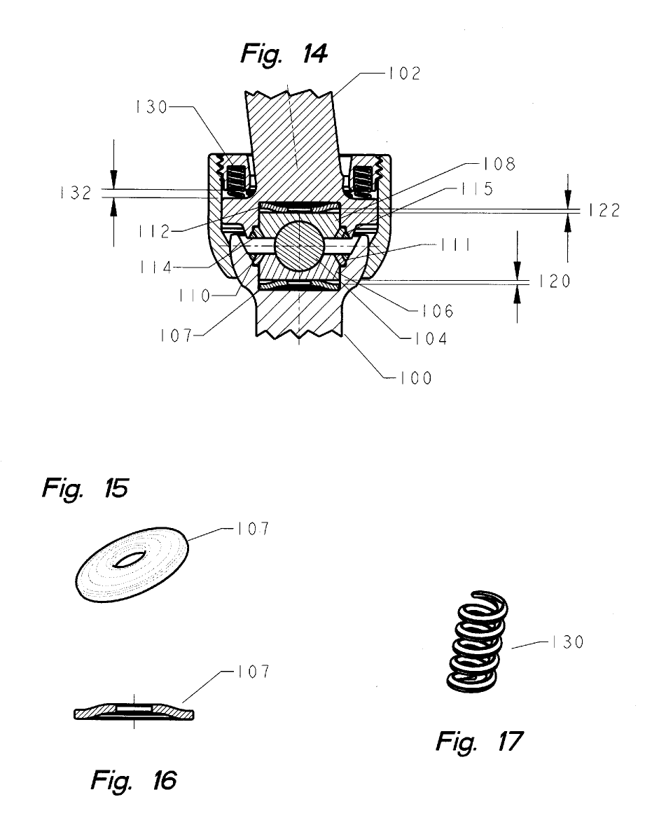

FIGS. 14 through 17 show a second embodiment utilizing coil springs and flexible spring washers in place of the polymeric O-rings of the previous embodiments. It should be noted that any combination of o-rings and spring washers may be used to accomplish the same purpose without departing from the scope of the invention. In this embodiment lower rod (100) is coupled to second rod (102) by use of a central ball bearing (104) having ball seat (106 and 108). Ball seat (106) is biased by spring washer (107) held in place by retaining ring (111) and weldment (110). Similarly, ball seat (108) flexible washer (112) all of which is held in place by retaining ring (115) and weldment (114). Ball seat (106) has a movement depicted by (120) similarly flexible washer (112) and can be compressed in accordance with the predetermined range of movement depicted by (122). Springs (130) allow for predetermined distraction and pivotal flexation of the rods as depicted by (132).

It should be understood that while we have illustrated and described certain forms of our invention it is not to be limited to the specific forms or arrangement of parts herein described and shown. It will be apparent to those skilled in the art that various changes can be made without departing from the scope of the invention and the invention is not to be considered limited to what is shown in the drawings and described in the specification.

What is claimed is:

1. A dynamic rod for use in posterior pedicle fixation that allows for load sharing across vertebrae by providing compression, distraction and bending comprising:
a first rod, a second rod and a flexible union for connecting the first and second rod,
said first rod having an enlarged end portion and said second rod having an enlarged end portion,
said flexible union including a cup member being generally cylindrical in shape and having a first open end and a second open end,
said enlarged portion of the first rod is larger than the first open end of the cup member and said enlarged portion of said first rod bears against an inner surface of the cup member,
said enlarged portion of the second rod is smaller than the second open end of the cup member and said enlarged portion of the second rod bears against an inner surface of the cup member, said union also including a retaining element secured to said second opening of the cup member to prevent the enlarged portion of the second rod from disengagement with the cup member,
said flexible union further including a bearing element interposed between a first bearing seat for the bearing element and a second bearing seat for the bearing element and,
a first resilient member interposed between the first bearing seat and an end face surface of the enlarged portion of the first rod and
a second resilient member interposed between the second bearing seat and an end face surface of the enlarged portion of the second rod.

2. A dynamic rod of claim 1 wherein said enlarged portion of the second rod has a second surface facing in a direction opposite to said end face surface on the second rod and a third resilient member in interposed between said second surface and said retainer member.

3. A dynamic rod of claim 1 wherein said first bearing seat is held in place by a first retaining ring and said second bearing seat is held in place by a second retaining ring.

4. A dynamic rod of claim 1 wherein said retaining element is threaded into the cap member and secured with a weld.

5. A dynamic rod of claim 4 wherein said weld is a laser weld.

6. A dynamic rod of claim 1 wherein said first resilient member is a polymeric O-ring.

7. A dynamic rod of claim 1 wherein said first resilient member is a flexible spring washer.

8. A dynamic rod of claim 1 wherein said second resilient member is a polymeric O-ring.

9. A dynamic rod of claim 1 wherein said second resilient member is a flexible spring washer.

10. A dynamic rod of claim 2 wherein said third resilient member is a polymeric O-ring.

11. A dynamic rod of claim 2 wherein said third resilient member is comprised of a plurality of coil springs.
